(19) United States
(12) Patent Application Publication
(10) Pub. No.: US 2023/0172522 A1
(43) Pub. Date: Jun. 8, 2023

(54) PROVIDING MENTAL CONTROL OF POSITION AND/OR GESTURE CONTROLLED TECHNOLOGIES VIA INTENDED POSTURES

(71) Applicants: BROWN UNIVERSITY, Providence, RI (US); THE GENERAL HOSPITAL CORPORATION, Boston, MA (US); The United States Government as Represented by the Department of Veterans Affairs, Washington, DC (US)

(72) Inventors: John D. Simeral, Greenwich, RI (US); Thomas Hosman, Providence, RI (US); Carlos Vargas-Irwin, Providence, RI (US); Daniel Thengone, Providence, RI (US); Leigh Hochberg, Providence, RI (US); Tyler Singer-Clark, Falmouth, MA (US)

(21) Appl. No.: 18/075,811

(22) Filed: Dec. 6, 2022

Related U.S. Application Data
(60) Provisional application No. 63/286,300, filed on Dec. 6, 2021.

(51) Int. Cl. A61B 5/375; G06F 3/01; A61B 5/384; A61B 5/00; A61B 5/11

(52) U.S. Cl. CPC A61B 5/375; G06F 3/015; A61B 5/384; A61B 5/7267; A61B 5/1116

(57) ABSTRACT

Neural signals of a subject intending certain postures can be decoded and a controllable device can be commanded to perform certain actions based on the decoded intended postures with a system, and method of use thereof, including a brain machine interface (BMI) device. The system also includes electrodes in communication with the subject's nervous system to record the neural signals and the controllable device, both in communication with the BMI device. The BMI device can store instructions and previously calibrated neural activity patterns for the certain postures and a processor for receiving the neural signals, pre-processing the neural signals, decoding the neural signals into neural activity patterns, and matching the neural activity patterns to the previously calibrated neural activity patterns. If a match is determined, then the BMI device can send a command, previously linked to the intended posture, to the controllable device to perform the action.

### RELATED APPLICATIONS

This application claims priority to U.S. Provisional Application Ser. No. 63/286,300, filed Dec. 6, 2021, entitled “BRAIN COMPUTER INTERFACE (BCI) SYSTEM THAT CAN BE IMPLEMENTED ON MULTIPLE DEVICES”. The entirety of this application is hereby incorporated by reference for all purposes.

### GOVERNMENT FUNDING STATEMENT

The present invention was made with government support under Grant No. NIDCD U01 DC017844 awarded by the National Institutes of Health and Grant No. A2295R awarded by the U.S. Department of Veterans Affairs. The US government has certain rights in this invention.

### TECHNICAL FIELD

The present disclosure relates to position and/or gesture controlled technologies, and more specifically, to a brain machine interface (BMI) device that can use a subject's intended posture(s) to provide mental control of the position and/or gesture controlled technologies.

### BACKGROUND

Recently, posture and/or gesture controlled technology has been increasing in prevalence in all parts of everyday life, from tablets, laptops, and phones to cars, refrigerators, faucets, lights, and even toasters. Such touch-controlled technologies rely on a controllable-device recognizing physical gestures and/or postures of a user to control actions. While gestures and postures are intuitive and easy to use for many able-bodied users, disabled users often struggle to, or simply cannot, form the gestures and/or postures required to act as inputs to the controllable devices, leaving a portion of the population cut off from access to common technologies.

### SUMMARY

Broader access to posture and/or gesture controlled technologies (for one or more controllable devices within the broader category of posture and/or gesture controlled technologies) can be provided through mental control. The systems and methods described herein relate to a user imagining performing one or more posture inputs to a controllable device and a brain machine interface (BMI) decoding neural signals related to the imagined posture and matching the intended posture with the associated input to the controllable device. The associated input can be sent from the BMI to the controllable device, enabling mental control.

In one aspect, the present disclosure includes a system for mentally controlling a controllable device. The system includes a plurality of electrodes, each configured to detect a neural signal within a nervous system (e.g., a brain) of a subject; a controllable device; and a brain machine interface (BMI) device in communication with the plurality of electrodes and the controllable device. The BMI device includes a non-transitory memory configured to store instructions and a plurality of previously calibrated neural activity patterns of the subject intending at least one predetermined posture; and a processor configured to implement the instructions. The instructions include: receive the neural signals from the plurality of electrodes; preprocess the neural signals; scan the preprocessed neural signals to detect a neural activity pattern; determine whether the neural activity pattern is indicative of the subject intending at least one predetermined posture by probabilistically matching the neural activity pattern to at least one previously calibrated neural activity pattern of the subject intending at least one predetermined posture of the plurality of previously calibrated neural activity patterns of the subject intending the at least one predetermined posture; and if the neural activity pattern is indicative of the subject intending the at least one predetermined posture, send a command to the controllable device to perform an action based on the subject intending the at least one predetermined posture. The controllable device performs the action upon receiving the command.

In another aspect, the present disclosure includes a method for mentally controlling a controllable device with a brain machine interface (BMI) device. The BMI device, which includes a processor, receives neural signals from a plurality of electrodes, wherein each of the plurality of electrodes are configured to detect the neural signals from nervous system (e.g., a brain) of a subject and to communicate with the BMI device. The BMI device preprocesses the neural signals and then scans the preprocessed neural signals to detect a neural activity pattern of the subject. The BMI device then determines whether the neural activity pattern is indicative of the subject intending at least one predetermined posture by probabilistically matching the neural activity pattern to at least one previously calibrated neural activity pattern of the subject intending at least one predetermined posture of a plurality of previously calibrated neural activity patterns of the subject intending at least one predetermined posture. If the neural activity pattern is indicative of the subject intending the at least one predetermined posture, then the BMI device sends a command, previously linked to the intended posture, to a controllable device to perform an action based on the subject intending the at least one predetermined posture. Upon receiving the command from the BMI device, the controllable device performs the action.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 shows a system for mentally controlling a controllable device;

FIG. 2 shows the BMI device of FIG. 1;

FIG. 3 shows an example use of the system of FIG. 1 for calibration of the detection of neural patterns within neural signals to create a posture profile;

FIG. 4 shows an example of the Posture Profile that can be stored in the memory of FIG. 1;

FIG. 5 shows an example use of the system of FIG. 1 for matching an intended posture to create an intended command;

FIG. 6 shows an example process flow diagram of a method for mentally controlling a controllable device;

FIG. 7 shows an example process flow diagram of a method for calibrating neural signals corresponding to an intended posture with an intended control;

FIG. 8 shows an example process flow diagram of a method for controlling a controllable device based on neural signals;

FIG. 9 shows an example process flow diagram of a method for probabilistically matching neural signals to output a specific command; and

FIG. 10 shows an example diagram of components of the system of FIG. 1 implementing the method of FIG. 6.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “posture and/or gesture controlled technology” refers to one or more devices with the ability to recognize or interpret positions, poses, or movements of one or more portions of a user's body as an input to a control a controllable device or part of a larger system in communication with the controllable device. Non-limiting examples of posture and/or gesture controlled technologies include touch-controlled interfaces, motion controlled interfaces, sound controlled technologies, or the like.

As used herein, the term “mental control” refers to employing a brain-machine interface to detect a subject's one or more intended actions via neural signals to be used in place of a physical postures and/or gestures in posture and/or gesture controlled technologies or one or more controllable devices within the broader category of posture and/or gesture controlled technologies. The term mental control generally means a computerized/controllable action performed based on the detection of mental/neural activity related to intended actions.

As used herein, the term “posture” refers to a fixed, static position (that does not rely on velocity) of at least a portion of a user's body (e.g., the user's body, limb, extremity, appendage, face, or the like) in space at a given time. For example, a hand posture can include specific held position of at least one of the hand, the wrist, or at least one finger (e.g., a held position of a thumbs up, a thumbs down, a fist, a flexed finger, an extended finger, or the like.). In another example, a facial posture can include the held position of a lifted eyebrow or a raised corner of a mouth. A static posture is distinct from a gesture, which is not static and relies on velocity (e.g., a gesture can include the act of swiping a finger to the left, right, up, or down, while a posture can include only a position at the beginning or end of the swipe). Multiple postures at different given times may be sequentially combined together to represent, convey, or the like, a gesture, without necessarily iterating the full path of movement, for example swiping left to right can be represented by pointing left and then pointing right.

As used herein, the terms “intended posture” and “imagined posture” can be used interchangeably herein to refer to a user's thought of making the user's body act in a certain way (assume/hold a certain posture), regardless of whether the body actually acts in the certain way in response to the thought. The intended posture may be used as an input to a controllable device through a brain machine interface (BMI).

As used herein, the term “brain machine interface (BMI)” refers to a device or system (including at least one non-transitory memory and at least one processor) that enables communication between a user's nervous system (e.g., brain) and a controllable device. The BMI can acquire neural signals (e.g., via one or more electrodes), analyze the neural signals (e.g., to detect/decode a neural activity pattern indicative of an intended posture), and translate the neural activity pattern into commands that are related to the controllable device (e.g., based on a posture profile for the user stored in memory). One example of a BMI is a Brain Computer Interface (BCI).

As used herein, the term “controllable device” refers to any device that can receive a command signal and then complete an action based on the command signal. Examples of controllable devices include, but are not limited to, a computer, a tablet, a mobile device, an environmental control element, a speech activation system, a robotic device, a prosthetic (e.g., for an arm, leg, hand, etc.), a soft robot, or the like. The controllable device, in some instances, can be part of a larger apparatus and/or posture and/or gesture controlled technology and can provide control of at least a portion of the larger apparatus and/or posture and/or gesture controlled technology.

As used herein, the terms “user” and “subject” can be used interchangeably to refer to any person, or animal, that can transmit neural signals to the BMI device. The person can be, for example, an individual with at least partial paralysis, a caregiver for an individual with paralysis, an individual missing at least part of a limb or extremity, an able-bodied individual, or the like. Another user can also refer to an additional person (e.g., a caregiver, technician, etc.) who may or may not be connected to the BMI device via electrodes.

As used herein, the term “electrodes” refers to one or more conductors used to transmit an electrical signal (e.g., transmitting neural signals from a user's brain to a BMI). For example, electrodes can be on or against the skull (e.g., electroencephalography EEG) electrodes or the like), near the brain (e.g., electrocorticography (ECoG) electrodes, any electrodes recording neural signals from blood vessels on or in the brain, or the like), and/or implanted in the brain (e.g., intracortical electrodes, deep brain electrodes, or the like). In some instances, two or more electrodes can be part of an array.

As used herein, the term “neural signals” refers to electrical signals generated by and recorded from a user's nervous system (e.g., at least a portion of the brain, like the cerebral cortex) by one or more electrodes and transmitted to a BMI. A plurality of electrodes can record an array of neural signals.

As used herein, the term “neural activity pattern” refers to at least a portion of one or more neural signals comprising recognizable neural features, such as threshold crossings and local field potential (e.g., spike band power), indicative of a specific thought of a subject, which can include an intended posture.

As used herein, the term “real time” refers to a time period, within 100 milliseconds, 50 milliseconds, 10 milliseconds, or the like, that seems virtually immediate to a user. For example, an input (neural signals) can be processed within several milliseconds so that the output (control signal) is available virtually immediately.

### II. Overview

Traditionally, an able-bodied user can use postures and/or gestures as inputs to a controllable device, but in certain circumstances, users (e.g., medically compromised and/or able bodied) are unable to use such postures and/or gestures as inputs. Accordingly described herein is brain machine interface (BMI) that enables a user to mentally control inputs of a controllable device (the inputs can engage the full range of movements a user can perform). Generally, BMI devices connect a subject with a device (such as a computer), often under the guidance of a caretaker or secondary operator and generally enable the subject to use neural activity to control a cursor without physical use of a computer mouse, joystick, or the like. Electrodes in, on, or near neural tissue of the subject are used to record neural signals of the subject connected to the BMI device, the neural signals are then used to control a controllable device. However, as technology advances controllable devices have grown from basic computers to include, for example, touch sensitive devices (e.g., tablets, mobile devices, etc.), complex robotic machines, prosthetic limbs, or the like that require more complex and nuanced control than traditional systems can provide. For example, many human-computer interfaces are designed to respond to touch actions that cannot be easily or intuitively achieved from actions achieved using a computer mouse. In another example, robotic machines or prosthetic limbs can include grasping mechanisms with complex multi-dimensional and multi-jointed control that cannot be achieved from simple computer mouse commands. As described herein, the BMI device can provide for more complex and expanded control based on intended postures of a subject.

Simply, the subject can imagine performing a posture and the BMI device can detect and use a user's intended/imagined postures as inputs to the controllable device. Virtual, otherwise called intended or imagined, postures are a static position of at least one part of a body in space at a time, can be used alone (e.g., to mimic a “posture” alone), in combination (e.g., to create a unique input control), or sequentially (e.g., to more easily mimic a gesture with a sequential number of posture), and can be a nearly unlimited pool to choose from. The BMI device can decode neural signals of the intended posture(s) and link the intended posture(s) (natural but virtual) to one of the inputs to the controllable device. The decoding of a large set of natural but virtual postures, such as hand postures, can create a BMI system that enables mental control of complex interfaces, such as a virtual “touch” interface. For example, with just finger, hand, and wrist postures as many as 40 or 50 or more commands can be reliably distinguished in real time (e.g., less than 100 ms latency).

### II. System

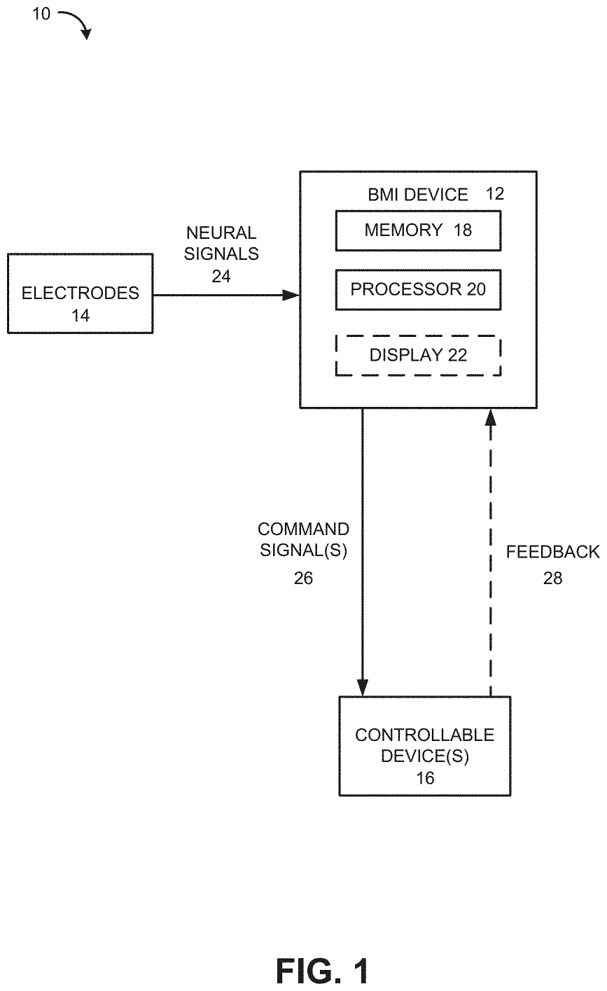

Many controllable devices use some form of posture and/or gesture controlled technology, posture and/or gesture controlled technologies as a whole operate according to inputs that are based on a subject physically making a particular posture and/or gesture. However, in certain circumstances, users (e.g., medically compromised and/or able bodied) are unable to use such physical postures and/or gestures as inputs. Provided herein is a system 10 (FIG. 1) that can use a BMI device 12 to enable a subject to mentally command at least one controllable device (which may be within a posture and/or gesture controlled technology) and/or the posture and/or gesture controlled technology as a whole (referred to collectively as controllable device(s) 16) to perform an action based on an intended posture.

The system 10 can include a plurality of electrodes (electrodes 14) to record neural signals of a subject's nervous system (e.g., a brain of a subject), a brain machine interface (BMI) device 12 that can decode intended postures from the neural signals into command signals 26 to control actions of at least one controllable device (controllable device 16), and the at least one controllable device that performs the actions. The BMI device 12 can be in communication with one or more of the electrodes 14, to receive the neural signals 24, and the controllable device 16, to send command signal(s) 26 to and optionally receive feedback 28 from the controllable device. The communication between the BMI device 12 and the electrodes 14 and/or the controllable device 16 can be wired and/or wireless (e.g., WIFI, Bluetooth, etc.) in any combination thereof. The BMI device 12 can include a non-transitory memory (memory 18) and a processor 20. The BMI device 12 can also include a display 22 that can be integrated with the BMI device or external to/separate from the BMI device but in communication, wired or wireless, with the BMI device. It should be understood that a brain of a subject is described herein, but the BMI devices can be operational with any one or more parts of a subject's nervous system.

Each of the electrodes 14 can detect and record neural signals 24 from the brain of the subject and send the neural signals to the BMI device 12. The electrodes 14 can each be positioned on and/or implanted into the brain of the subject. The electrodes 14 may be on the skull (e.g., electroencephalography (EEG) electrodes or the like), near the brain (e.g., electrocorticography (ECoG) electrodes, any electrodes recording neural signals from blood vessels on or in the brain, or the like), and/or implanted in the brain (e.g., intracortical electrodes, deep brain electrodes, or the like). The electrodes 14 can, for example, be positioned on and/or implanted into the left precentral gyrus of the brain of the subject to detect and record neural signals 24 at least related to intended/imagined hand postures (e.g., postures of a hand, a wrist, and/or at least one finger). In one example, the electrodes 14 can be at least one multi-channel intracortical microelectrode array positioned on and/or implanted into the brain. For example, two 96-channel intracortical microelectrode arrays can be chronically implanted into the precentral gyms of the subject's brain. In another example, the electrodes may also be implanted and/or surface electrodes able to record from a portion of the subject's peripheral nervous system (e.g., for an amputee). The electrodes 14 can be connected to the controllable device by a wired connection, a wireless connection, or an at least partially wired and wireless connection.

The controllable device (one or more of controllable device(s) 16) can receive command signals 26 (e.g., one or more command signals) from the BMI device 12 (over a wired connection, a wireless connection, or an at least partially wired and wireless connection) and perform one or more actions in response to receiving the command signals. The controllable device 16 may also send feedback data (feedback 28) (e.g., data related to an aspect of the controllable device, data related to the action performed by the controllable device, etc.) back to the BMI device 12. A single controllable device 16 is shown in FIG. 1, and throughout, but it should be understood that the controllable device can be more than one controllable device (e.g., two or more that are commanded in response to different intended postures) and/or that the subject, or another user of the system 10 (e.g., a caregiver, an assistant, a medical professional, or the like), can choose to switch what controllable device is being controlled through the BMI device 12 at any given time. The switch between controllable devices 16 can be through a specific predetermined intended posture of the subject and/or manually by the subject or another user of the system. The controllable device(s) 16 can include a controller, a memory and processor, or any other control circuitry necessary to receive command signals and then execute actions, and optionally send feedback to the BMI device 12. Examples of controllable devices 16 include, but are not limited to a computer, a tablet, a mobile device, an environmental control element, a speech activation system, a robotic device, a prosthetic, or a soft robot. For example, the controllable device 16 can be a work system, in the environment of the subject or remote from the subject, such as a manufacturing robot putting together a product, a computer running a software (e.g., word processing, computational, CAD, or the like), a computerized train (e.g., where the subject is a train engineer), an elevator (e.g., where the subject is a concierge). In another example, the controllable device 16 can be an environmental control element, such as a motorized wheelchair, a smart piece of furniture, a smart thermostat, smart lightbulb, security/safety devices (e.g., cameras, alarms, or the like), or the like. Where smart refers to any objects that include circuits and/or computer components that can cause an action.

The controllable device 16 can be a device that is natively commanded by a touch interface (such as a touch screen tablet or track pad). However, mouse-enabled point-and-click can be a limited or ineffective control input for modern computers and mobile devices that are designed with powerful touch and gesture-based interfaces. This is particularly true for BMI users with severe motor disability who can be highly reliant on wheelchairs and the powerful mobile devices that can be mounted on them, such as smart phones or tablets. In such a case at least one predetermined posture can directly replace at least one of the native gesture and/or touch commands of the device. For example, if the controllable device 16 has a touch screen that responds to swiping left and/or right to change the screen, then the intended posture can include intending to point a finger left and/or right to command the screen to change.

In some instances, the non-transitory memories (such as memory 18 of the BMI device 12) and the processors (such as processor 20 of the BMI device) can be hardware devices. Software aspects that can be implemented by the associated devices can be stored as computer program instructions in the non-transitory memories. The non-transitory memories can each be any non-transitory medium that can contain or store the computer program instructions, including, but not limited to, a portable computer diskette; a random-access memory; a read-only memory; an erasable programmable read-only memory (or Flash memory); and a portable compact disc read-only memory). The computer program instructions may be executed by the processors. The one or more processors can each be one or more processors of a general-purpose computer, special purpose computer, and/or other programmable data processing apparatus. Upon execution of the computer program instructions, various functions/acts can be implemented.

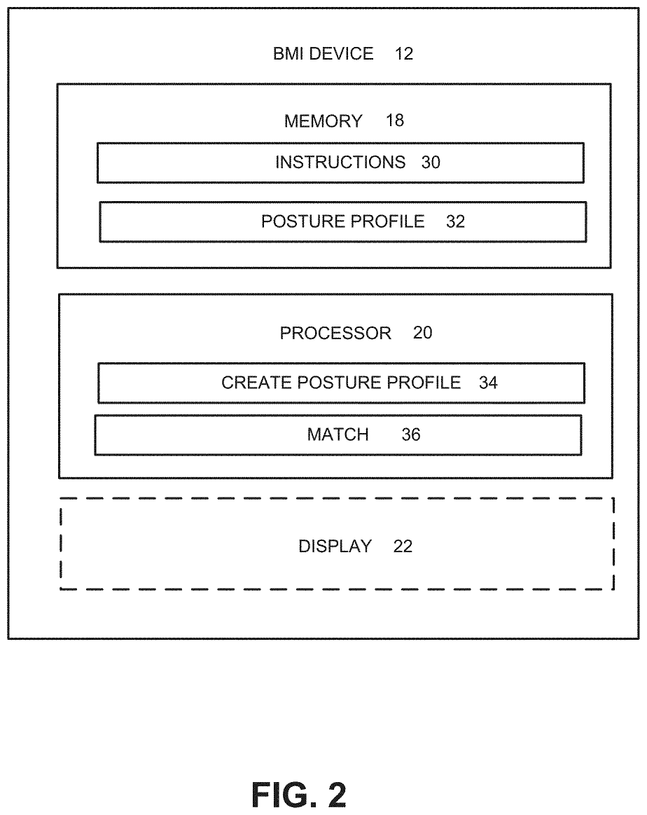

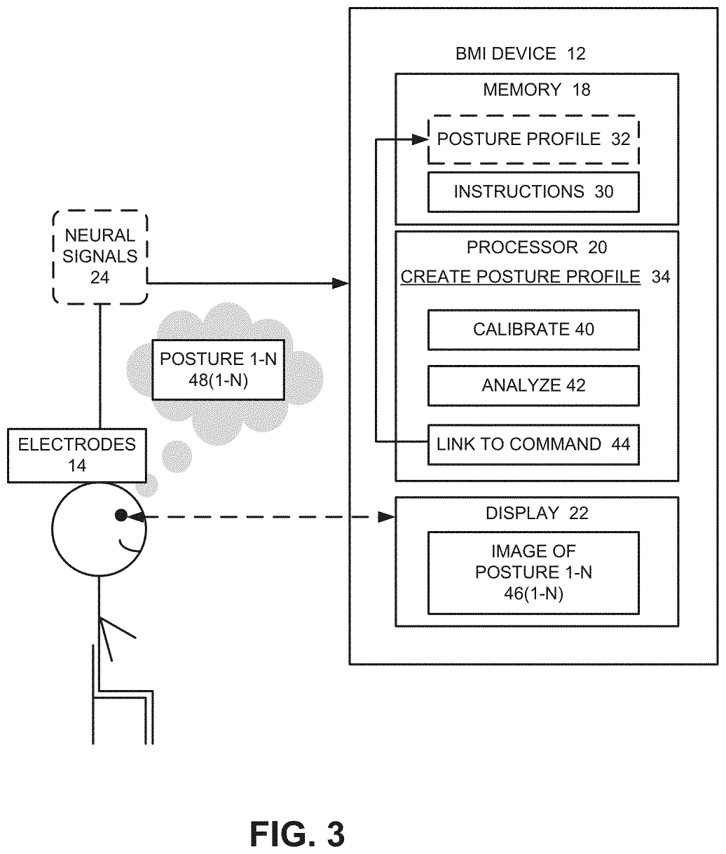

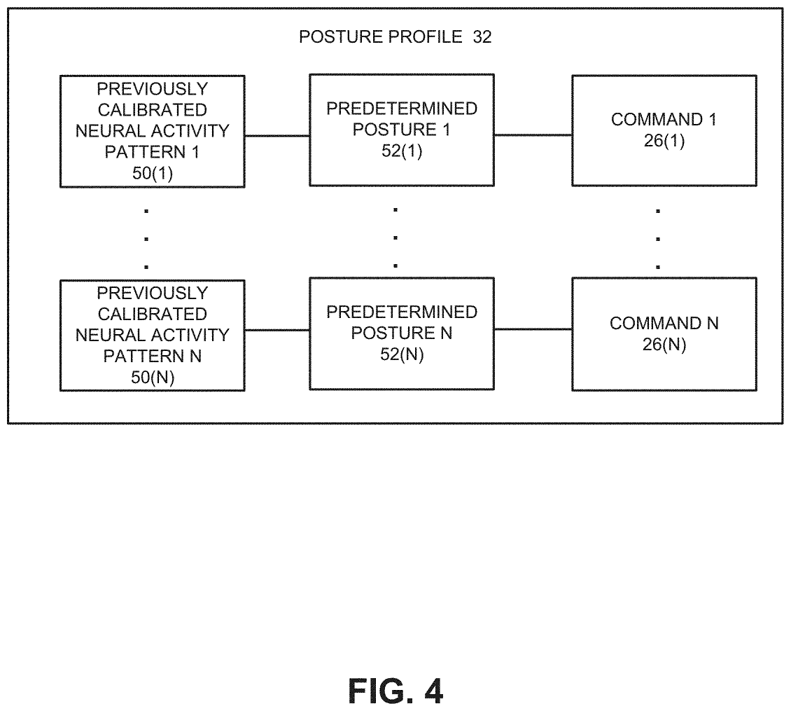

FIG. 2 shows the BMI device 12 in greater detail. The memory 18 can store instructions 30, including, but not limited to, instructions for calibrating the BMI device 12 and creating a subject specific Posture Profile and instructions for decoding intended postures to control the controllable device(s). The memory 18 can also store the subject specific posture profile 32, which can include a plurality of previously calibrated neural activity patterns of the subject intending at least one predetermined posture and is explained in greater detail below with reference to FIGS. 3-4. The processor 20 can implement the instructions to calibrate the BMI device and create the subject specific Posture Profile 34 and the instructions to match the neural signals with an at least one predetermined posture 36 to command the controllable device. The instructions 30 stored in the memory 18 and implemented by processor 20 may also include a traditional cursor and click decoder that can be used in conjunction with the gesture decoder. The BMI device may include a display 22 that can be integral to the BMI device or in communication (wired or wireless) with the BMI device. The display 22 can include a visual and an audio component. The display 22 can output visuals and/or audio for calibration, creation of the Posture Profile, feedback from the controllable device(s), a BMI specific graphical user interface, confirmation screens. and/or any other information relevant to use of the BMI device 12. The display 22 and/or the BMI device 12 may also be linked to a traditional user input device (e.g., a mouse, keyboard, touchscreen, or the like) that an able-bodied user can use to help set up, fix, or make changes to the BMI device 12.

FIG. 3 illustrates an example use of the system 10 to calibrate the BMI device 12 and create a posture profile for a subject to use the BMI device. The electrodes 14 can be in, on, or near the brain of the subject (e.g., in electrical communication) and in communication with the BMI device 12. The BMI device 12 can include the memory 18, the processor 20, and the display 22, as described previously. The memory 18 can store instructions 30 and the subject specific posture profile 30 after it is created. The instructions 30 can include instructions to create a posture profile 34 that are implemented by the processor 20. The instructions to create the posture profile 34 can begin with instructions to calibrate 40 the BMI device for the specific subject. Calibration can include the display 22 of the BMI device 12 showing the subject one or more images of desired postures 1-N 46(1-N). Calibration may alternatively and/or additionally include audibly and/or tacitly instruct the subject of the desired postures 1-N. As an image of each posture 1-N 46(1-N) is shown to the subject, the subject can be instructed to intend to assume the posture shown on the screen. For each image of posture 1-N 46(1-N) the neural signals 24 of the subject can be detected and recorded by the electrodes 14. The neural signals 24 can include neural features, such as local field potential features and thresholded action potential features. The BMI device 12 can receive the neural signals 24 continuously, including when the subject intends each specified posture. The image of the same posture can be repeated as many times as necessary to achieve a confident calibration. For each set of neural signals 24 received (e.g., per posture image) the processor 20 can then analyze and process 42 the neural signals 24 received each time a specific posture image 1-N 46(1-N) is shown to the user to create an aggregated and confident neural activity pattern of the subject that is indicative of the subject intending that posture. The analysis and processing of the of the neural signals 24 can include preprocessing of the raw data, generating specific neural features of interest, z-scoring the neural features of interest, smoothing the neural features of interest, and selecting and combining the features. This can be done for each repetition of the same intended posture and aggregated until the BMI device has a certain confidence level in the neural activity pattern of the subject being indicative of the subject intending that posture. Each neural activity pattern can then be linked to a specific command 44 of the subject's choosing for a controllable device of the subject's choosing. In a Posture Profile 32 the subject may link the same calibrated intended posture with different commands (or the same command) for different controllable devices. It should be noted that calibration can be separated from creation of the Posture profile if one or more intended postures needs to be recalibrated or a subject chooses to change what intended posture is linked with what command.

FIG. 4 shows an example Posture Profile 32 that is stored in the memory of the BMI device. The Posture Profile can include any number of previously calibrated neural activity patterns 1-N 50(1)-50(N). Each of the previously calibrated neural activity patterns 1-N 50(1)-50(N) is indicative of a predetermined intended posture 1-N 52(1)-52(N) (e.g., the image shown on the display during the calibration process) and linked to a specific command 1-N 26(1)-26(N) (e.g., command signal) that can be sent to a specific controllable device when the controllable device is in communication with the BMI device 12. It should be understood that the postures may in fact be completed physically by the subject but may be only mental, as the BMI device decodes the neural signals generated in the subject's brain by the intention, not the visual movement or the muscular activation or the like.

Each of the predetermined postures that the subject intends can be a fixed position of at least one body part in space at a time. The postures can be at least one specific intended position of a body, a limb, one or more extremities, one or more appendages, or a part of a face of the subject. A hand posture, as an example can include a position of at least one of a hand, a wrist, and at least one of the fingers on the hand. For example, a finger pointed right, left, up, or down, a thumbs up, a thumbs down, a peace sign, the ok sign, or the like are each different postures of the hand. In some instances, a posture can include two or more postures in sequence or in combination. For example, a sequential posture can include making a first at a first time, then a thumbs up a certain time later. In another example, a combination posture can include pointing right with a finger of the left hand and making the peace sign with the right hand at the same time. A user may choose to use only a subset of all possible postures in order to improve decoding accuracy during use of the BMI device. A multi-state decoder method is specifically used to detect the intention of natural postures (e.g., hand postures such as swipes, grasps, finger movements, peace sign, or the like) and map the intended posture to commands for computer-controllable interactions. The posture decoding can be applied, in one example, to control tactile (touch) interfaces. For example, a touch and swipe enabled tablet can be controlled using intended postures even when actual touch is not possible. The decoder can be trained to distinguish when one of the intended postures is commanded by the user (relative to no action intended) and to distinguish which one of the postures is intended at any given moment.

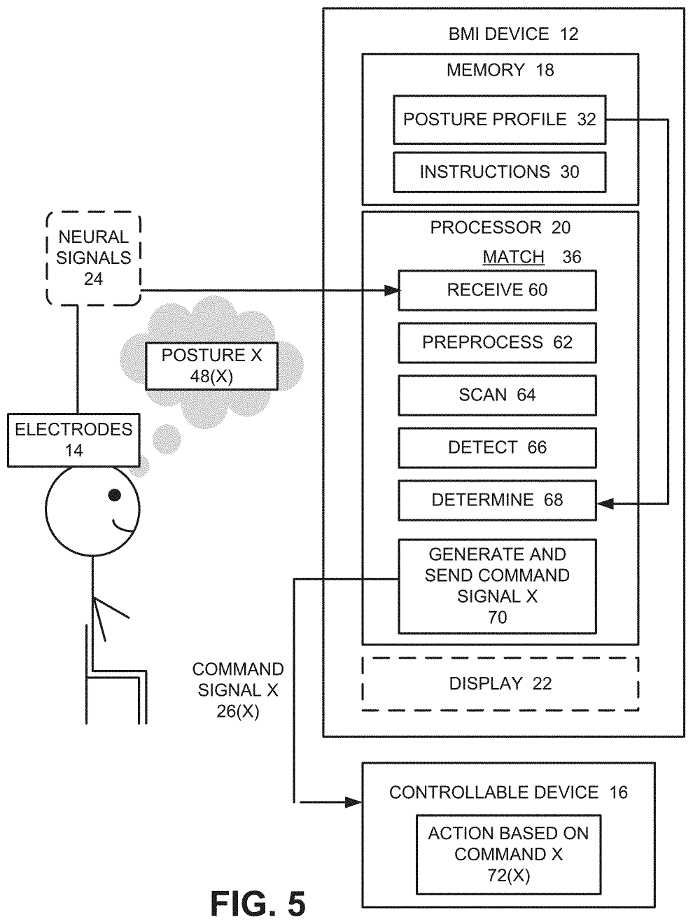

Referring again to FIG. 2, the BMI device 12 can execute a match program 36 with the processor 20 (shown in FIG. 5). The match program 26 compares portions of the neural signals to the posture profile to identify an intended command. The BMI device 12 can decode neural signals 24 of the intended posture and link the intended posture (natural but virtual) to one of the inputs to the controllable device 16. The decoding of a large set of natural but virtual postures, such as hand postures, can create a BMI system that enables mental control of complex interfaces, such as a virtual “touch” interface.

FIG. 5 illustrates a use of the system 10 to match an intended posture of a subject to input a command on a controllable device 16. The BMI device 12 is in electrical communication (wired and/or wireless) with the electrodes 14 and the controllable device 16. When the subject decides they want to make the controllable device 16 perform a given action 72(X) the subject intends a specific posture X 48(X). The electrodes 14 detect and record the neural signals 24 of the subject as the subject intends the specific posture X 48(X). Neural signals 24 can include neural features such as action potential features, local field potential features, or any other type of feature that can be compared between neural signals. The BMI device 12 can include the memory storing the subject specific Posture Profile 32 (an example of which is shown in FIG. 4) and the instructions 30, which can include the match instructions 36 that are implemented by the processor 20. The BMI device 12 can receive 60 the neural signals 24 from the electrodes 14. The BMI device 12 can continuously receive, in real time, the neural signals 24 from the electrodes 14 while the device is actively running a decoding program (when the user is actively connected to the BMI device 12), regardless of if the user is intending a posture or not. The neural signals 24 can be preprocessed 62 by the BMI device. As a non-limiting example, the pre-processing can include multiple steps to filter and clean the data including, but not limited to the following. First raw data of the neural signals 24 can be filtered and excess noise can be removed. For example, the data can be downsampled to 15 Ksps with a finite impulse response (FIR) filter, then a common average reference (CAR) can be applied to each data grouping (e.g., array) to remove large common signals (e.g., electrical noise), and then a bandpass filter can be applied to bandpass the data between 250 Hz and 5000 Hz. Second, the neural features of the preprocessed raw data can be generated, specifically threshold crossings and local field potential for each data set. The threshold crossings are events when the signal goes below the electrode's 3.5 RMS value. Local field potential is the total filtered data's power (sum of squared data) for the time step (20 ms). Next, the neural features can be z-scored over time. The BMI device 12 can track the means and variances of the neural features over time and then normalize each feature per electrode by subtracting the mean and dividing by the square root of the variance, or the standard deviation. After the z-scoring, the BMI device 12 can smooth the features with a rolling average window between 0 (no smoothing) and 1 second of the previous feature's value. Finally, the BMI device 12 can select and combine specific channels of the threshold crossings and local field potential neural features to form the preprocessed neural signals to pass on to the scan functionality.

The BMI device 12 can then scan 64 the preprocessed neural signals and detect 66 any neural activity pattern (that may be indicative of an intended posture) in the neural signals. As an example, the detection can be based on general classes of known neural feature combinations for the previously calibrated intended postures. The BMI device 12 can then determine whether the neural activity pattern is indicative of the subject intending at least one predetermined posture by probabilistically matching the neural activity pattern to at least one previously calibrated neural activity pattern of the subject intending at least one predetermined posture of the plurality of previously calibrated neural activity patterns of the subject intending the at least one predetermined posture. For example, when the subject is intending/imagining posture X 48(X) the BMI device probabilistically matches the neural activity pattern of the subject as they are intending posture X with each of the previously calibrated neural activity patterns 1-N 50(1)-50(N) in the Posture Profile to determine that the subject is actually intending posture X 48(X). The probabilistic matching can include using a machine learning based multi-state decoder model such as a linear discriminant analysis combined with a hidden Markov model or a recurrent neural network. The hidden Markov model can be used to determine transitions between posture states. For example, the determination can include, but is not limited to computing probabilities by comparing the current selected neural features to per-class (i.e., posture) averages and covariances estimates for each of the neural activity patterns for the previously calibrated intended postures for every time step. Then the class probabilities can be normalized such that the sum of all probabilities adds to 1, optionally the probabilities can be post processed by smoothing the probabilities with a rolling average window from 0 (no smoothing) to one second. A class may be selected if the class probability is above a predetermined class threshold (e.g., 0.85, 0.9, 0.95, or the like). If configured to decode a single posture, only the largest class probability is considered, otherwise, each posture probability is thresholded. If no class is above the threshold, then the BMI device defaults to no posture was intended so no command signal is generated or sent.

The BMI device 12 can, in some instances, query the subject (e.g., via visual or audio means) to determine if the correct intended posture has been matched. If the neural activity pattern is indicative of the subject intending the at least one predetermined posture (in this case posture X 48(X)), then the BMI device 12 can generate the command linked with the predetermined posture and send the command to the controllable device 16 to perform an action 72 based on the subject intending the at least one predetermined posture. The controllable device 16 can perform the action 72 based on the command signal as an input in response to the intended posture. If no neural activity pattern indicative of the subject intending the at least one predetermined posture is matched (or the answer to the BMI device's 12 query is answered in the negative) then the matching instructions continue through the neural signals from the next time period.

Additionally, intended postures can be used to control the BMI device 12 itself. For example, a specially chosen posture can be used to initiate calibration if control has degraded to the point where calibration cannot be selected with cursor control on the computer screen or if a certain number of incorrect decodes occur in a time period. In another example, a specially chosen posture can pause neural decoding (except for future recognition of the un-pause gesture) so that the user can prevent accidental control of the controllable device while simply reading a page of text on a computer screen or watching a video. In a further example, a special posture can be chosen so that the user can switch BMI control from a first controllable device to a second controllable device.

### IV. Method

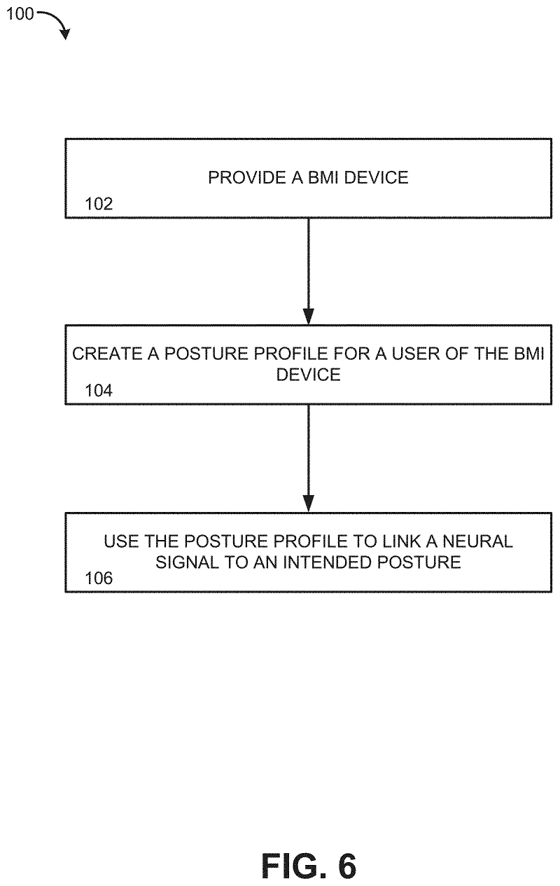

Another aspect of the present disclosure can include methods (FIGS. 6-10) that relate to calibration and use of a brain machine interface to decode imagined postures into inputs for a controllable device. The methods can be executed by the system 10 of FIGS. 1-5. The methods 100, 200, 300, and 400 are illustrated as process flow diagrams with flowchart illustrations that can be implemented by one or more components of the system 10. The method 500 is an example of the components of the system 10 implementing the method of FIG. 6 illustrated as a flow diagram. It should be understood that a brain of a subject is described herein, but the BMI devices can be operational with any one or more parts of a subject's nervous system.

For purposes of simplicity, the methods 100, 200, 300, 400, and 500 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 100, 200, 300, 400, and 500.

FIG. 6 illustrates a method 100 for a user mentally controlling a controllable device with an intended posture taking the place of a physical posture and/or gesture. The controllable device can be, for example, a computer, a tablet, a mobile device, an environmental control element, a speech activation system, a robotic device, a prosthetic, or a soft robot. The method 100 can be particularly useful for controlling a controllable device that is normally touch controlled, where traditional BMIs fail to enable usability for patients with motor or limb-based disorders or infirmities. At 102, a BMI Device is provided, such as BMI device 12 of system 10. The BMI device is in communication with at least one controllable device and a plurality of electrodes positioned in, on, or near the brain of the user of the BMI device. The BMI device includes, but is not limited to, a non-transitory memory for storing instructions and a Posture Profile and a processor for implementing the instructions and can include a display that can be integral with the device or in electrical communication with the BMI device. The BMI device can be configured to decode neural signals indicative of predetermined intended postures to send command signals to be input into the at least one controllable device to make the at least one controllable device perform actions. At 104, the Posture Profile, which can be stored in the memory of the BMI device, is created for the user of the BMI device. Creating the posture profile can include calibrating the BMI device to recognize the neural activity patterns in the user's neural signals that indicate one or more predetermined intended postures, linking each of the neural activity patterns to the predetermined intended posture, and then matching each of the neural activity patterns for each of the predetermined intended postures with a specific command to make a specific controllable device perform a specific action of the user's choosing. At 106, after the user's posture profile is completed, the Posture Profile can be used to match a neural signal of the user at a given time to an intended posture to input a command to the controllable device such that the user can mentally control the controllable device.

For example, postures reminiscent of gestures associated with touch interfaces (such as a tablet) are imagined by the user to enable touch-like control of the touch-enabled device interfaces. For example, an imagined, virtual wrist or finger flexed upwards can be mapped to a swipe-up operating system call on the target device (to scroll up in a window, for example). However, the imagined posture need not mimic an actual able-bodied touch action.

Thus, in another example, any imagined posture can be mapped to any function of interest on the target computer or device. The intent to make a closed first posture can be mapped to open a Windows context menu as if a right-click had been performed. In another example, decoded postures can be assigned to achieve novel computer actions when software is placed on the device to receive and interpret posture commands that are not natively understood by the device. For example, an imagined open palm gesture detected in the neural signals can be decoded and mapped to a text-to-speech function that generates a “Hello” voice output from a speech providing device.

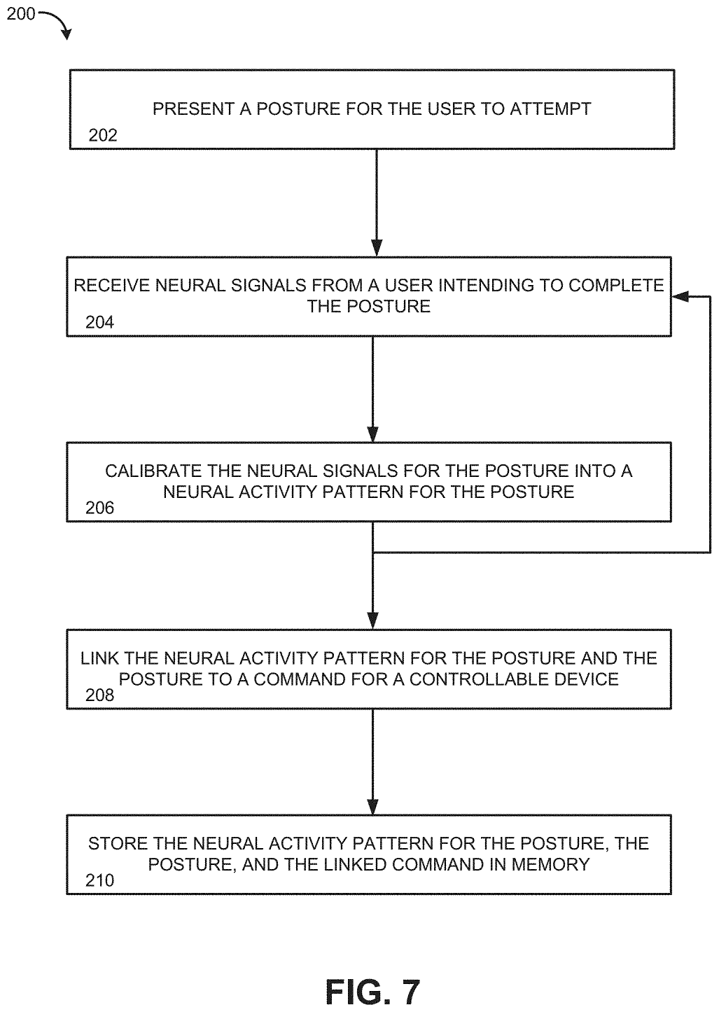

FIG. 7 illustrates a method 200 for calibrating neural signals corresponding to an intended postures and creating the Posture Profile linking the calibrated neural signals for intended postures with command signals for input to the one or more controllable devices (or re-linking previously calibrated postures to new/different commands). At 202, the BMI device (via an integral or connected display) requests a posture for the user to input. For example, the BMI device can present the posture to the user. The posture can be presented to the user as at least one of an image, text, audio, visual description, or the like. Example postures presented to the user can include, but are not limited to, point left, point right, thumbs up, peace sign, smile, lift eyebrow, wink, or the like. The presentation of the posture can be highlighted on the display in any manner (e.g., entering a circle, moving towards a line, going from black and white to color, growing larger, growing louder if audio, or the like) to indicate that the user should begin intending to complete the posture. At 204, the BMI device can receive the neural signals from the user, via the plurality of electrodes, as the user is intending to assume the posture. The user can intend to assume the posture, e.g., think about making the posture in real life, regardless of if the posture can be completed in real life due to a disability or infirmity. At 206, the neural signals of the user intending the posture can be calibrated into a neural activity pattern for the posture. The neural activity pattern for a given intended posture can be calibrated on multiple iterations of steps 202 and 204 that are processed, analyzed, and aggregated together to create a confident neural activity pattern for that given intended posture with known neural features. Once a confident neural activity pattern for a given intended posture is calibrated, at 208, that neural activity pattern for the intended posture and the intended posture are linked to a command signal that can act as an input for a controllable device. The BMI device can enable the user to choose what controllable device and what input command each neural activity pattern of an intended posture is linked to. The same neural activity pattern of an intended posture can be linked to different command signals for different controllable devices. For example, the user can intend a thumbs up to control a smart thermostat to increase temperature and to control a volume to increase on a smart tv, where the destination of the control signal can depend on what controllable device is actively connected to the BMI device at a time. At 210, the neural activity pattern for the intended posture, the posture, and the linked command can be stored in the memory of the BMI device as the Posture Profile to be accessed when the BMI device is in use for mentally controlling the controllable device. Any number of neural activity patterns for intended postures, postures, and linked commands can be stored in the Posture Profile. The Posture Profile can be updated at any time to include different postures, different controllable devices, and/or different command signals.

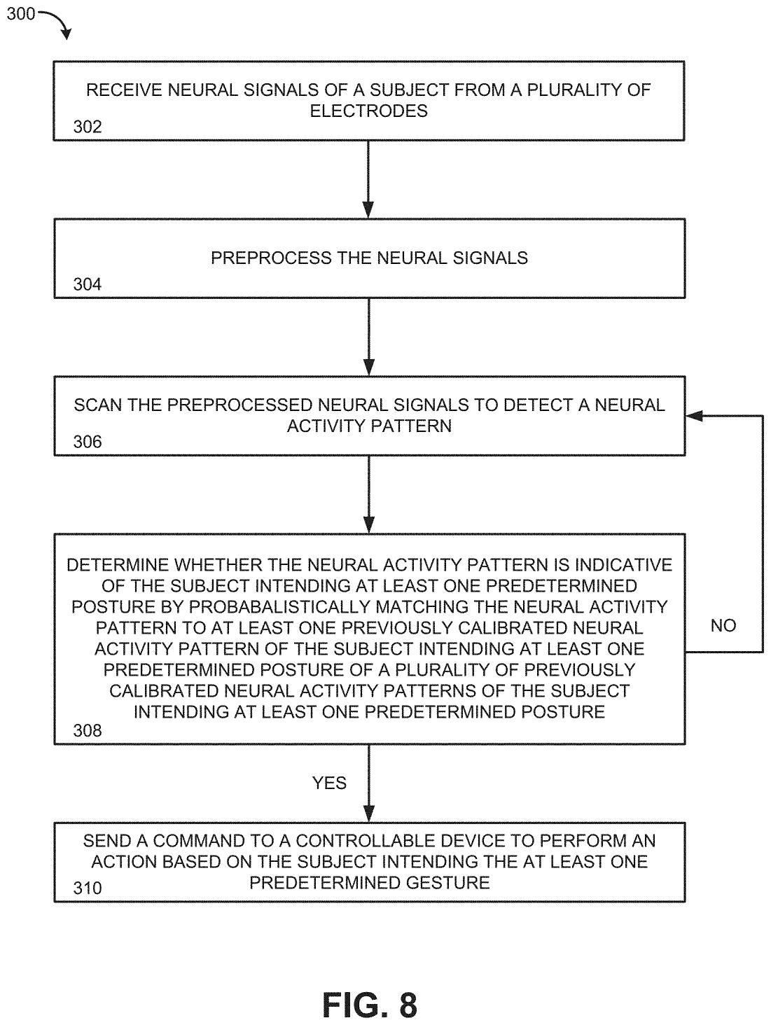

FIG. 8 illustrates a method 300 for controlling a controllable device based on neural signals indicative of the user intending at least one predetermined (calibrated) posture. At 302, the BMI device receives neural signals from the plurality of electrodes, wherein each of the plurality of electrodes are configured to detect the neural signals from the brain of the subject and to communicate with the BMI device. At 304, the BMI device can preprocess the neural signals, as described in detail above to filter the raw neural signals and remove noise. At 306, the BMI device can scan the preprocessed neural signals to detect a neural activity pattern of the subject (e.g., if the subject is not thinking of intending a posture of any sort, then no neural activity pattern may be detectable). At 308, the BMI device can determine whether the neural activity pattern is indicative of the subject intending at least one predetermined posture by probabilistically matching the neural activity pattern to at least one previously calibrated neural activity pattern of the subject intending at least one predetermined posture of a plurality of previously calibrated neural activity patterns of the subject intending at least one predetermined posture. If no match indicative of the subject intending at least one predetermined intended posture it is determined that the BMI device can return to scanning the preprocessed neural signals for the next time period. If the neural activity pattern is indicative of the subject intending at least one predetermined posture, then at 310, the BMI device can send a command to a controllable device to perform an action based on the subject intending the at least one predetermined posture.

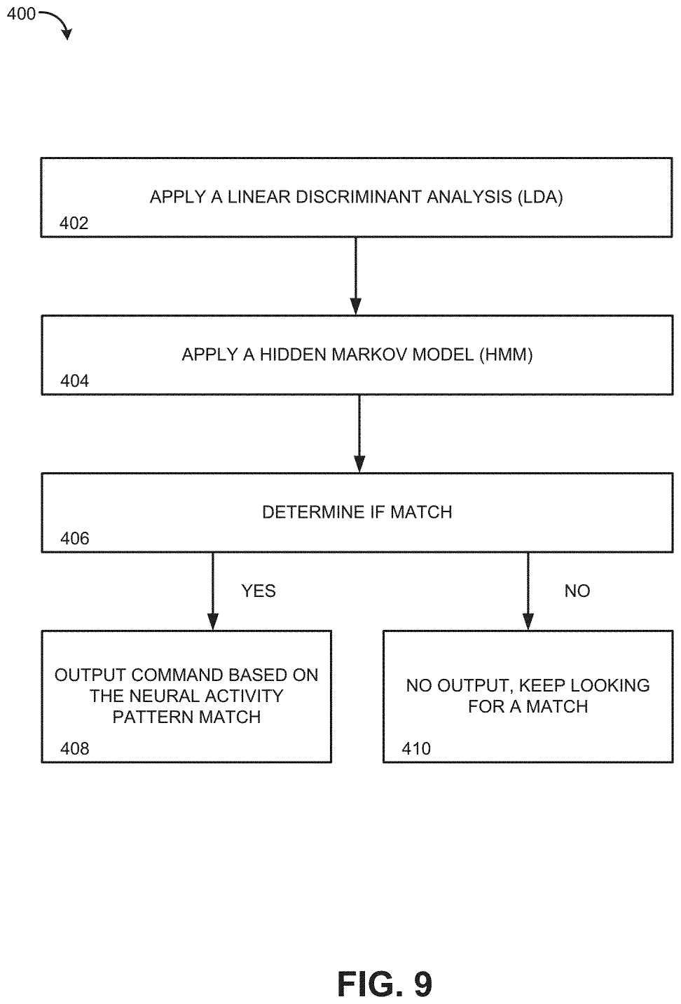

FIG. 9 illustrates a method 400 for probabilistically matching neural signals to output a specific command to be used as an input for a controllable device to perform a specific action. It should be noted that FIG. 9 shows one possible method for probabilistically matching, but other methods can be used (e.g., using a recurrent neural network). At 402, a linear discriminant analysis can be applied to the neural activity pattern. Then at step 404, a hidden Markov model can be applied to the neural activity pattern to probabilistically match the neural activity pattern with the previously calibrated neural activity patterns stored in the Posture Profile. At 406, the BMI device can determine if the neural activity pattern matches one of the previously calibrated neural activity patterns with a high enough confidence level based on a threshold (described in greater detail above). If the BMI device determines there is a match, then at 408 the BMI device outputs a command signal (to be sent to the controllable device) based on the neural activity pattern match. The specific command signal outputted is the command signal linked to the intended posture and the neural activity pattern of the intended posture in the Posture Profile. If the BMI determines no there is not a match (the probabilities are below their respective posture threshold), then at 410 the BMI device outputs no command signals and continues to decode the neural signals and look for a match.

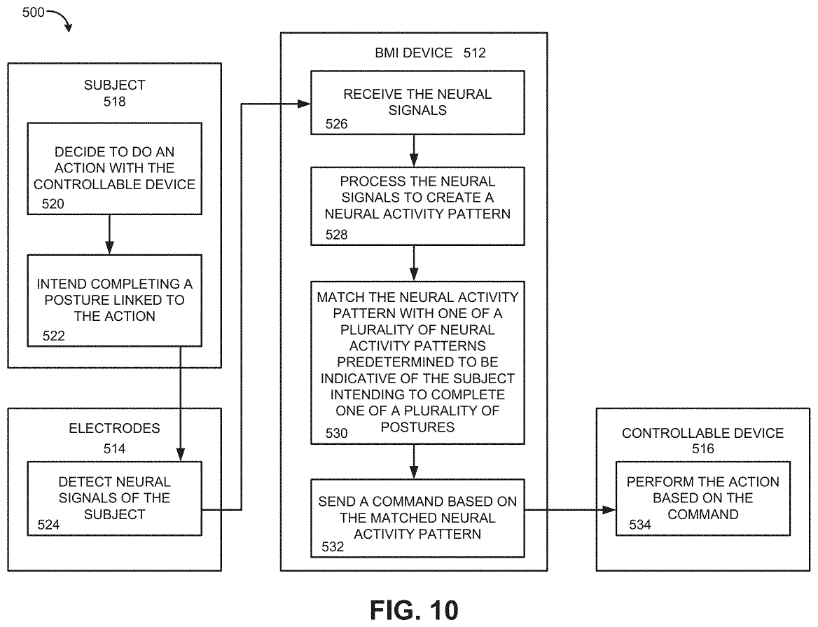

FIG. 10 illustrates an example implementation 500 of the system 10 for mentally controlling the controllable device 516 with an intended posture. The BMI device 512 is in electrical communication (wired and/or wireless) with the electrodes 514 and the controllable device 516. The electrodes 514 are in, on, and/or near the brain of the subject 518 and in communications with the brain of the subject. At 520, the subject decides that they want the controllable device 516 to perform an action. At 522, the subject intends to assume a posture (regardless of if the posture is actually made in the physical world) that has previously been linked to the action of the controllable device the user wants to have happen. At 524, the electrodes 514 detect and record the neural signals of the subject 518. The electrodes 514 continuously detect and record the neural signals of the subject 518, including as the subject intends the posture. At 526, the neural signals are received by the BMI device 512 from the electrodes 524. At 528, the BMI device processes the neural signals, preprocessing and scanning, to represent the neural activity pattern of the user (at a time). At 530, the BMI device probabilistically matches the neural activity pattern (at the time) with the plurality of neural activity patterns predetermined (during calibration and stored in the Posture Profile) to be indicative of the subject intending to complete a predetermined posture. When the BMI device has matched the neural activity pattern with the neural activity pattern predetermined to be indicative of the subject intending to complete a predetermined posture (and optionally the BMI device has checked that the correct posture was determined), then at 532 the BMI device can generate the command based on the matched neural activity pattern and associated with the intended posture (stored in the Posture Profile) and send the command to the controllable device 516. At 534, the controllable device 516 can, after receiving the command signal as an input, perform the action based on the command signal. Thus, the subject 518 can control the controllable device mentally via intended postures.

From the above description, those skilled in the art will perceive improvements, changes, and modifications. Such improvements, changes and modifications are within the skill of one in the art and are intended to be covered by the appended claims. All patents, patent applications, and publications cited herein are incorporated by reference in their entirety.

What is claimed is:

1. A system comprising:
a plurality of electrodes, each configured to detect a neural signal within a nervous system of a subject;
a controllable device; and
a brain machine interface (BMI) device in communication with the plurality of electrodes and the controllable device, the BMI device comprising:
a non-transitory memory configured to store instructions and a plurality of previously calibrated neural activity patterns of the subject intending at least one predetermined posture; and
a processor configured to implement the instructions to:
receive the neural signals from the plurality of electrodes;
preprocess the neural signals;
scan the preprocessed neural signals to detect a neural activity pattern;
determine whether the neural activity pattern is indicative of the subject intending at least one predetermined posture by probabilistically matching the neural activity pattern to at least one previously calibrated neural activity pattern of the subject intending at least one predetermined posture of the plurality of previously calibrated neural activity patterns of the subject intending the at least one predetermined posture; and
if the neural activity pattern is indicative of the subject intending the at least one predetermined posture, send a command to the controllable device to perform an action based on the subject intending the at least one predetermined posture.

2. The system of claim 1, wherein the at least one predetermined posture is a fixed position of at least one body part in space at a time.

3. The system of claim 1, wherein the at least one predetermined posture comprises at least one specific intended position of a body, a limb, one or more extremities, one or more appendages, or a part of a face of the subject.

4. The system of claim 1, wherein the controllable device is at least one of a computer, a tablet, a mobile device, an environmental control element, a speech activation system, a robotic device, a prosthetic, or a soft robot.

5. The system of claim 1, wherein the at least one predetermined posture replaces at least one native gesture command of the controllable device.

6. The system of claim 1, wherein the plurality of electrodes are each configured to be positioned on and/or implanted into the left precentral gyrus of the brain of the subject.

7. The system of claim 1, wherein the plurality of electrodes comprises at least one multi-channel intracortical microelectrode array.

8. The system of claim 1, wherein the neural signals comprise action potential features, local field potential features, or one or more features derived from the neural signals.

9. The system of claim 1, wherein the probabilistic matching further comprises using a machine learning based multi-state decoder model.

10. The system of claim 9, wherein the machine learning based multi-state decoder model comprises a linear discriminant analysis combined with a hidden Markov model or a recurrent neural network.

11. The system of claim 1, wherein the processor further executes the instructions to create a Posture Profile of the subject for the controllable device in the non-transitory memory.

12. The system of claim 11, wherein creating the Posture Profile comprises:
linking each of the stored plurality of previously calibrated neural activity patterns of the subject intending at least one predetermined posture with a specific command for the controllable device to perform a specific action.

13. The system of claim 1, wherein the processor further executes the instructions to calibrate the BMI device.

14. The system of claim 13, wherein calibration of the BMI device comprises:
displaying, on a display associated with the system, a plurality of postures;
detecting and recording, via the plurality of electrodes, the neural activity of the subject's brain when the subject intends each of the plurality of postures as each of the plurality of postures are displayed.

15. A method comprising:
receiving, by a Brain Machine Interface (BMI) device comprising a processor, neural signals from a plurality of electrodes, wherein each of the plurality of electrodes are configured to detect the neural signals from a nervous system of a subject and to communicate with the BMI device;
preprocessing, by the BMI device, the neural signals;
scanning, by the BMI device, the preprocessed neural signals to detect a neural activity pattern of the subject;
determining, by the BMI device, whether the neural activity pattern is indicative of the subject intending at least one predetermined posture by probabilistically matching the neural activity pattern to at least one previously calibrated neural activity pattern of the subject intending at least one predetermined posture of a plurality of previously calibrated neural activity patterns of the subject intending at least one predetermined posture; and
if the neural activity pattern is indicative of the subject intending the at least one predetermined posture, sending, by the BMI device, a command to a controllable device to perform an action based on the subject intending the at least one predetermined posture.

16. The method of claim 15, wherein the at least one predetermined posture comprises a fixed position of at least one body part of the subject in space at a time.

17. The method of claim 15, wherein the at least one predetermined posture comprises at least one specific intended position of a body, a limb, one or more extremities, one or more appendages, or a face of the subject.

18. The method of claim 15, wherein the probabilistic matching further comprises using a machine learning based multi-state decoder model.

19. The method of claim 15, further comprising:
creating, by the BMI device, a Posture Profile of the subject for the controllable device in the non-transitory memory, wherein the Posture Profile comprises:
the plurality of previously calibrated neural activity patterns of the subject intending at least one predetermined posture; and
a specific command for the controllable device to perform a specific action matched with each of the plurality of previously calibrated neural activity patterns of the subject intending the at least one predetermined posture.

20. The method of claim 19, further comprising calibrating the BMI device by:
displaying, on a display associated with the system, a plurality of postures; and
detecting and recording, via the plurality of electrodes, the neural activity of the subject's brain when the subject intends each of the plurality of postures as each of the plurality of postures are displayed.
